SEQUENCE LISTING (23 sequences)

SEQ ID NO: 1 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIARNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 2 - DNA, 1137 bp, Bacillus subtilis
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgcccgtaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 3 - protein, 353 aa, E. coli
MLYTSQTTPEKDQKMSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 4 - protein, 349 aa, E. coli
MKIKAVGAYSAKQPLEPMDITRREPGPNDVKIEIAYCGVCHSDLHQVRSEWAGTVYPCVPGHEIVGRVVAVGDQVEKYAPGDLVGVGCIVDSCKHCEECEDGLENYCDHMTGTYNSPTPDEPGHTLGGYSQQIVVHERYVLRIRHPQEQLAAVAPLLCAGITTYSPLRHWQAGPGKKVGVVGIGGLGHMGIKLAHAMGAHVVAFTTSEAKREAAKALGADEVVNSRNADEMAAHLKSFDFILNTVAAPHNLDDFTTLLKRDGTMTLVGAPATPHKSPEVFNLIMKRRAIAGSMIGGIPETQEMLDFCAEHGIVADIEMIRADQINEAYERMLRGDVKYRFVIDNRTLTD

SEQ ID NO: 5 - DNA, 789 bp, Ralstonia sp.
atggctagcagaggatcgcatcaccatcaccatcacggcgcctatcgactattaaacaaaacagccgtcataaccggtggaaacagcggcattggcctcgccacagcgaagcgcttcgttgccgagggtgcctatgtattcattgtcggtcgccggcggaaggaactcgagcaggcggccgcagaaatcggtcggaatgtcacggcggtcaaagccgatgtgacaaagcttgaagacctggaccgactttacgcgattgtgcgtgagcaacggggtagcatcgacgtactatttgcgaattccggcgcaatcgagcaaaagacgcttgaggagattactccggaacactatgacaggactttcgatgtcaacgttcggggattgatcttcaccgtgcagaaggcacttcctctgctgcgagacggcggcagcgtgatcctgacaagctcggtagccggcgtcctaggattacaggcgcacgacacgtatagtgccgccaaggcagcggtaaggtcgctcgcgaggacatggaccactgagttgaaaggtcgcagcattcgtgtcaacgcggtaagcccaggggcgatcgacacgcctatcatagaaaaccaggtctctacacaggaagaagctgacgagctgcgtgcgaaatttgcagctgcgacgcccctgggtcgcgtcggacgacctgaagagctggcagcggccgtgttatttcttgcatcggacgacagtagctacgtagccggcattgagctgtttgtggacggtggattgacccaggtctaa

SEQ ID NO: 6 - protein, 459 aa, Chromobacterium violaceum ATCC 12472
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 7 - protein, 452 aa, Pseudomonas putida
MSEQNSQTQAWQALSRDHHLAPFSDVKQLAEKGPRIITSAKGVYLWDSEGNKILDGMAGLWCVAVGYGRDELAEVASQQMKQLPYYNLFFQTAHPPALELAKAIADVAPQGMNHVFFTGSGSEGNDTVLRMVRHYWALKGKKNKNVIIGRINGYHGSTVAGAALGGMSGMHQQGGVIPDIVHIPQPYWFGEGGDMTEADFGVWAAEQLEKKILEVGVDNVAAFIAEPIQGAGGVIIPPQTYWPKVKEILARYDILFVADEVICGFGRTGEWFGTDYYDLKPDLMTIAKGLTSGYIPMGGVIVRDEVAKVISEGGDFNHGFTYSGHPVAAAVGLENLRILRDEQIIQQVHDKTAPYLQQRLRELADHPLVGEVRGLGMLGAIELVKDKATRARYEGKGVGMICRQHCFDNGLIMRAVGDTMIIAPPLVISVEEIDELVQKARKCLDLTYEAVR

SEQ ID NO: 8 - protein, 452 aa, Pseudomonas putida; identical to SEQ ID NO: 7

SEQ ID NO: 9 - protein, 453 aa, Pseudomonas putida
MSVNNPQTREWQTLSGEHHLAPFSDYKQLKEKGPRIITKAQGVHLWDSEGHKILDGMAGLWCVAVGYGREELVQAAEKQMRELPYYNLFFQTAHPPALELAKAITDVAPQGMTHVFFTGSGSEGNDTVLRMVRHYWALKGKPHKQTIIGRINGYHGSTFAGACLGGMSGMHEQGGLPIPGIVHIPQPYWFGEGGDMTPDAFGVWAAEQLEKKILEVGEDNVAAFIAEPIQGAGGVIIPPETYWPKVKEILAKYDILFVADEVICGFGRTGEWFGSDYYDLKPDLMTIAKGLTSGYIPMGGVIVRDTVAKVISEGGDFNHGFTYSGHPVAAAVGLENLRILRDEKIVEKARTEAAPYLQKRLRELQDHPLVGEVRGLGMLGAIELVKDKATRSRYEGKGVGMICRTFCFDNGLIMRAVGDTMIIAPPLVISHAEIDELVEKARKCLDLTLEAIN

SEQ ID NO: 10 - protein, 455 aa, Rhodobacter spaeroides
MRDDAPNSWESRADASSFYGFTDLPSVHQRGTVVLTHGKGPYIYDVHGRAYLDANSGLWNMVAGFDHPGLIEAAKAQYERFPGYHAFFGRMSDQTVMLSEKLVEVSPFARGRVFYTNSGSEANDTMVKMLWFLGAAEGHPERRKIITRVNSYHGVTAVSASMTGKPYNSLFGLPLPGFIHVGCPHYWRFGQAGETEAEFTQRLARELEATIIKEGPDTIAGFFAEPVMGAGGVIPPSEGYFQAVQPVLKRYGIPLIADEVICGFGRTGNTWGCQTYDFMPDGIISSKNITAGFFPMGAVILGPELADRLQAASEAVEEFPHGFTASGHPVGCAIALKAIDVVMNEGLAENVRALTPKFEAGLAYLAENPNIGEWRGKGLMGALEAVKDKATKTPFPGDLSVSERIANSCTDHGLICRPLGQSIVLCPPFIMTEAQMDEMFEKLGAALKKVFAEVA

SEQ ID NO: 11 - DNA, 23 bp, artificial
  primer
gccatcataggggttcctaaaga

SEQ ID NO: 12 - DNA, 30 bp, artificial
  primer
actgatggtaccttaagcacccgccacaga

SEQ ID NO: 13 - DNA, 33 bp, artificial
  primer
gtgacgatcattgcccgtaacgcagaccgcttg

SEQ ID NO: 14 - DNA, 33 bp, artificial
  primer
caagcggtctgcgttacgggcaatgatcgtcac

SEQ ID NO: 15 - DNA, 27 bp, artificial
  primer
gcctatcgactattaaacaaaacagcc

SEQ ID NO: 16 - DNA, 30 bp, artificial
  primer
actgatggtaccttagacctgggtcaatcc

SEQ ID NO: 17 - DNA, 1401 bp, Paracoccus denitrificans
atggctagcagaggatcgcatcaccatcaccatcacggcgccaaccaaccgcaaagctgggaagcccgggccgagacctattcgctctacggtttcaccgacatgccctcggtccatcagcggggcacggtcgtcgtgacccatggcgaggggccctatatcgtcgatgtccatggccgccgctatctggatgccaattcgggcctgtggaacatggtcgcgggcttcgaccacaagggcctgatcgaggccgccaaggcgcaatacgaccgctttcccggctatcacgcctttttcggccgcatgtccgaccagaccgtgatgctgtcggaaaagctggtcgaggtctcgccattcgacaacggccgggtcttctataccaattccggctccgaggcgaacgacaccatggtcaagatgctgtggttcctgcatgccgccgagggcaagccgcaaaagcgcaagatcctgacgcgctggaacgcctatcacggcgtgaccgcggtttcggcctcgatgaccggcaagccctacaactcggtcttcggcctgccgctgcccggcttcatccacctgacctgcccgcattactggcgctatggcgaggaaggcgagaccgaggcgcaattcgtcgcccgcctggcacgcgagcttgaggataccatcacccgcgagggcgccgacaccatcgccggcttcttcgccgagccggtgatgggcgcggggggggtgatcccgccggcgaagggttatttccaggccatcctgccgatcttgcgcaagtatgacatcccgatgatctcggacgaggtgatctgcggcttcgggcgcaccggcaacacctggggctgcctgacctacgacttcatgcccgatgcgatcatctcgtccaagaacctgactgcgggcttcttcccgatgggcgccgtcatcctcgggcccgacctcgccaagcgggtcgaggccgcggtcgaggcgatcgaggagttcccgcacggcttcaccgcctcgggccatccggtcggctgcgccatcgcgctgaaggccatcgacgtggtgatgaacgaggggctggccgagaatgtccgccgcctcgcaccccgcttcgaggcggggctgaagcgcatcgccgaccgcccgaacatcggcgaataccgcggcatcggcttcatgtgggcgctggaggcggtcaaggacaagccgaccaagacccccttcgacgccaatctttcggtcagcgagcgcatcgccaatacctgcaccgatctggggctgatctgccggccgctgggccagtccatcgtgctgtgcccgcccttcatcctgaccgaggcgcagatggacgagatgttcgaaaagctggaaaaggcgctcgacaaggtctttgccgaggtggcctaa

SEQ ID NO: 18 - DNA, 23 bp, artificial
  primer
gccaaccaaccgcaaagctggga

SEQ ID NO: 19 - DNA, 33 bp, artificial
  primer
actgataagcttttaggccacctcggcaaagac

SEQ ID NO: 20 - DNA, 24 bp, artificial
  primer
ctgccggccaatgggccagtccat

SEQ ID NO: 21 - DNA, 24 bp, artificial
  primer
atggactggcccattggccggcag

SEQ ID NO: 22 - protein, 391 aa, Artificial
  Bacillus subitilis protein mit His-Tag
MASRGSHHHHHHGAIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIARNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 23 - DNA, 1176 bp, artificial
  Bacillus subtilis protein mit His-Tag
atggctagcagaggatcgcatcaccatcaccatcacggcgccatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgcccgtaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

(19) United States
(12) Patent Application Publication
(10) Pub. No.: US 2015/0099282 A1
(43) Pub. Date: Apr. 9, 2015

(54) NADP-DEPENDENT ALANINE DEHYDROGENASE

(71) Applicants: Thomas Haas, Muenster (DE); Jan Christoph Pfeffer, Hanau (DE); Arne Skerra, Freising (DE); Alexandra Lerchner, Neufahrn bei Freising (DE); Alexander Jarasch, Muenchen (DE)

(72) Inventors: Thomas Haas, Muenster (DE); Jan Christoph Pfeffer, Hanau (DE); Arne Skerra, Freising (DE); Alexandra Lerchner, Neufahrn bei Freising (DE); Alexander Jarasch, Muenchen (DE)

(73) Assignee: EVONIK INDUSTRIES AG, Essen (DE)

(21) Appl. No.: 14/395,666

(22) PCT Filed: Apr. 16, 2013
(86) PCT No.: PCT/EP2013/057855

§ 371 Date: Oct. 20, 2014

(30) Foreign Application Priority Data
Apr. 20, 2012 (EP) 12164933.9

(51) Int. Cl. C12P 13/06; C12N 9/06

(52) U.S. Cl. CPC C12P 13/06; C12N 9/0016; C12Y 104/01001
USPC 435/116; 435/325; 435/252.3; 435/252.33; 435/254.2; 435/254.21; 435/254.22; 435/254.23

(57) ABSTRACT

The present invention relates to a polypeptide comprising the amino acid sequence of the Bacillus subtilis alanine dehydrogenase or a variant thereof, where Leu197 or an amino acid which is located at a homologous position in the amino acid sequence is exchanged for an amino acid with a positively charged side chain, to a nucleic acid molecule encoding such a polypeptide, and to a process for the production of alanine or a compound generated with consumption of alanine, comprising the step of reacting pyruvate with ammonium and NADPH to give alanine by bringing the pyruvate into contact with the polypeptide according to the invention or with the cell according to the invention.

The present invention relates to a polypeptide comprising the amino acid sequence of the Bacillus subtilis alanine dehydrogenase (AlaDH) or a variant thereof, where Leu197 or an amino acid which is located at a homologous position in the amino acid sequence is exchanged for an amino acid with a positively charged side chain, to a nucleic acid molecule encoding such a polypeptide, and to a process for the production of alanine or of a compound generated with consumption of alanine, comprising the step of reacting pyruvate with ammonium and NADPH to give alanine by bringing the pyruvate into contact with the polypeptide according to the invention or with the cell according to the invention.

Amines are used as synthesis units for a multiplicity of products of the chemical industry, such as epoxy resins, polyurethane foams, isocyanates and, in particular, polyamides. The latter are a class of polymers characterized by repeating amide groups. In contrast to the chemically related proteins, the expression “polyamides” usually refers to synthetic commercially available thermoplastics. Polyamides are derived from primary amines or from secondary amines which can be obtained from alkanes generated during the cracking of fossil hydrocarbons. However, amine derivatives, more precisely aminocarboxylic acid, lactams and diamines, may also be used for the preparation of amide-based polymers. Other interesting starting materials are short-chain gaseous alkanes which can be obtained from renewable raw materials by means of biotechnological processes and subsequently aminated.

Many polyamides for which there is a strong commercial demand are prepared starting from lactams. For example, “nylon-6” can be obtained by polymerizing ε-caprolactam, and “nylon-12” by polymerizing laurolactam. Other products of commercial interest comprise copolymers of lactam, for example copolymers of ε-caprolactam and laurolactam.

The traditional chemo-technical production of amines is dependent on the supply of fossil raw materials, inefficient and generates large amounts of undesirable by-products, in some synthetic steps up to 80%. An example of such a process is the preparation of laurolactam, which is traditionally obtained by trimerizing butadiene. The trimerization product cyclododecatriene is hydrogenated, and the resulting cyclododecane is oxidized to give cyclodecanone, which is subsequently reacted with hydroxylamine to give cyclododecane oxine, which is finally converted into laurolactam via a Beckmann rearrangement reaction.

In consideration of these disadvantages, processes were developed for obtaining amines from renewable raw materials, using biocatalysts. Here, agricultural products such as sugar or fatty acids, which may already comprise alcohol functions, are oxidized to give the aldehyde or ketone. In a subsequent step, the aldehyde or the ketone is subsequently converted into the amine by a transaminase, with consumption of an amino acid. The amino acid is advantageously regenerated by an amino acid dehydrogenase which is present during the transaminase reaction, with consumption of an inorganic nitrogen salt.

For example, the international patent application PCT/EP 2008/067447 describes a biological system for the preparation of ω-aminocarboxylic acids using a cell which includes a series of suitable enzymatic activities and which is capable of converting carboxylic acids into the corresponding ω-aminocarboxylic acid. In particular, the cell includes the AlkBGT oxidase system from Pseudomonas putida GPO1, which first oxidizes the ω-aminocarboxylic acid to give the ω-hydroxycarboxylic acid and then to give the aldehyde. This is followed by an amination of the oxidation product by a transaminase, which is also expressed.

EP11174729.1 and EP11006458.1 describe processes for the preparation of amines starting from alkanes or from alcohols, which processes are distinguished in that the alcohol, or the alkane which has been hydroxylated, in a first process step, to give the alcohol, is reacted by an alcohol dehydrogenase with consumption of NAD⁺ to give the aldehyde or ketone and the product subsequently reacted by a transaminase with consumption of alanine as the amine donor to give the amine. The alanine consumed can be regenerated by an alanine dehydrogenase which is present, with consumption of pyruvate and ammonium, so that, overall, alanine need not be supplied.

Among the alanine dehydrogenases described in the literature, the Bacillus subtilis alanine dehydrogenase and variants thereof are suitable for such a process. The disadvantage of this specific group of alanine dehydrogenases is that they are NADH-dependent, that is to say they consume another reduced redox factor than the one which the large numbers of NAD-dependent alcohol dehydrogenases which have proved to be especially advantageous in biotechnology generate during the oxidation of the alcohol.

In the case of an in vitro reaction, this means that one NAD⁺ molecule and one NADPH molecule must be supplied to the reaction per reacted alcohol molecule. If, in contrast, the reaction is carried out in vivo using a whole-cell catalyst, then the cell can, in principle, provide the respective redox factors via the primary metabolism; however, this is a considerable burden on the metabolism which entails a reduced product yield and/or a shorter service life of the cell.

Against this background, the object on which the invention is based is that of providing a redox-neutral enzyme system for the amination of alcohols, i.e. a system comprising enzymes which do not require the supply of externally produced NAD⁺ or NADPH or other redox factors for catalysing the conversion of alcohol into the amine with regeneration of the alanine.

A further object on which the invention is based consists in supplying an alanine dehydrogenase which is suitable for the enzymatically catalysed conversion of alcohol into the amine in the presence of NADPH and which catalyses the reaction with as high a turnover number, i.e. the number of substrate molecules reacted per second, as possible.

A further object on which the invention is based consists in modifying the Bacillus subtilis alanine dehydrogenase in such a way that the enzyme catalyses the conversion of pyruvate into alanine with a higher affinity to NADPH as the substrate and that the affinity of the enzyme for NADPH is preferably higher than that for NADH, more preferably without the result being significant reduction of the turnover number relative to the turnover number of the wild-type enzyme.

A further object on which the invention is based consists in providing a redox-neutral system for the amination of alcohols, which system is suitable for a one-pot reaction under mild reaction conditions, i.e. in particular those without extreme temperatures or pH values and in the absence of heavy-metal-containing catalysts or other toxic compounds.

These and further objects are achieved by the subject matter of the present application and in particular also by the subject matter of the appended independent claims, where embodiments can be found in the dependent claims.

In a first aspect, the problem on which the invention is based is solved by a polypeptide comprising the amino acid sequence of the Bacillus subtilis alanine dehydrogenase or a variant thereof, where Leu197 or an amino acid which is located at a homologous position in the amino acid sequence is exchanged for an amino acid with a positively charged side chain.

In a first embodiment of the first aspect, the problem is solved by a polypeptide, wherein the amino acid with positively charged side chain is arginine.

In a second embodiment of the first aspect, which is also an embodiment of the first aspect, the problem is solved by a polypeptide wherein additionally Asp196 or an amino acid located at a homologous position in the amino acid sequence is exchanged for an amino acid with a neutral or positively charged side chain.

In a third embodiment of the first aspect, which is also an embodiment of the first to second aspects, the problem is solved by a polypeptide, wherein the amino acid with a neutral or positively charged side chain is an amino acid from the group comprising alanine, glycine, serine and cysteine, preferably alanine.

In a fourth embodiment of the first aspect, which is also an embodiment of the first to second aspects, the problem is solved by a polypeptide which is the polypeptide shown in SEQ ID No. 1.

In a second aspect, the problem on which the invention is based is solved by a nucleic acid molecule comprising a nucleotide sequence encoding the polypeptide according to the first aspect and its embodiments.

In a first embodiment of the second aspect, the problem is solved by a nucleic acid molecule which is the nucleotide sequence shown in SEQ ID No. 2.

In a third aspect, the problem on which the invention is based is solved by a vector comprising the nucleic acid molecule according to the second aspect and its embodiments.

In a fourth aspect, the problem on which the invention is based is solved by a cell comprising the polypeptide according to the first aspect, the nucleic acid molecule according to the second aspect or the vector of the third aspect and the respective embodiments.

In a first embodiment of the fourth aspect, the problem is solved by a cell, which furthermore expresses an NADP⁺-dependent alcohol dehydrogenase.

In a second embodiment of the fourth aspect, which is also an embodiment of the first aspect, the problem is solved by a cell according to the fourth aspect or the first embodiment of the fourth aspect, which furthermore expresses a transaminase.

In a fourth aspect, the problem on which the invention is based is solved by a process for the production of alanine or of a compound generated with consumption of alanine, comprising the step of
c) reacting pyruvate with ammonium and NADPH to give alanine by bringing the pyruvate into contact with the polypeptide according to the first aspect or with the cell according to the second aspect or one of the respective embodiments.

In a first embodiment of the fourth aspect, the problem is solved by processes furthermore comprising
a) reacting a primary or secondary alcohol to give the aldehyde or ketone, respectively, by contacting the alcohol with an NADP⁺-dependent alcohol dehydrogenase in the presence of NADP⁺.

In a second embodiment of the fourth aspect, which is also an embodiment of the first aspect, the problem is solved by a process which furthermore comprises
b) reacting of the aldehyde or ketone prepared in step a) to give the amine by bringing the aldehyde or ketone into contact with a transaminase in the presence of alanine.

In a third embodiment of the fourth aspect, which is also an embodiment of the first to second aspects, the problem is solved by a process wherein steps a), b) and c) proceed in the same reaction mixture, preferably simultaneously.

In a further embodiment of the third or fourth aspect or in an embodiment of the third or fourth aspect, the problem is solved by a cell or by a process where the cell intracellularly expresses all enzymes from the group comprising the polypeptide according to one of claims 1 to 5, the NADP⁺-dependent alcohol dehydrogenase and the transaminase in a localized manner.

In a further embodiment of the third or fourth aspect or in an embodiment of the third or fourth aspect, the problem is solved by a cell or by a process wherein the NADP⁺-dependent alcohol dehydrogenase is an NADP⁺-dependent alcohol dehydrogenase from the group comprising the alcohol dehydrogenases from E. coli (YjgB, database code ZP_{—}07117674, and a YahK, database code BAE76108.1) and the alcohol dehydrogenase from Ralstonia sp. (SEQ ID No. 3), preferably the alcohol dehydrogenase from Ralstonia sp. (SEQ ID No. 5), or variants thereof.

In a further embodiment of the third or fourth aspect or in an embodiment of the third or fourth aspect, the problem is solved by a cell or by a process wherein the transaminase is a transaminase from the group comprising the transaminases from Chromobacterium violaceum (database code NP_{—}901695), Pseudomonas putida (database code YP_{—}001668026.1 or YP_{—}001671460) and Rhodobacter sphaeroides (strain ATCC 17023; database code YP_{—}353455) and variants thereof, preferably the transaminase from Chromobacterium violaceum (database code NP_{—}901695).

The present invention is based on the inventors' surprising finding that an enzyme system comprising an alcohol dehydrogenase, preferably an NADP⁺-dependent alcohol dehydrogenase, a transaminase and a polypeptide comprising the amino acid sequence of the Bacillus subtilis alanine dehydrogenase or a variant thereof, where Leu197 or an amino acid which is located at a homologous position in the amino acid sequence is exchanged for an amino acid with a positively charged side chain can be used for catalysing the amination of an alcohol in a redox-neutral fashion.

Furthermore, the present invention is based on the inventors' surprising finding that the affinity of a polypeptide, comprising the amino acid sequence of the Bacillus subtilis alanine dehydrogenase, for NADPH can be increased by Leu197 or an amino acid which is located at a homologous position in the amino acid sequence being exchanged for an amino acid with a positively charged side chain while the enzyme remains catalytically active and is capable of catalysing the reaction with a high turnover number.

The invention relates to a polypeptide comprising the amino acid sequence of the Bacillus subtilis alanine dehydrogenase or a variant thereof, where Leu197 or an amino acid which is located at a homologous position in the amino acid sequence is exchanged for an amino acid with a positively charged side chain. This polypeptide is shown in the sequence listing under SEQ ID No. 1. In a preferred embodiment, the expression “alanine dehydrogenase” as used in the present context is understood as meaning an enzymatically active polypeptide which catalyses the conversion of pyruvate into alanine and NAD⁺ or NADP⁺, preferably NADP⁺, with consumption of ammonium and NADH or NADPH, respectively, preferably NADPH. The amino acid sequence of the Bacillus subtilis alanine dehydrogenase, and also the sequences of the other molecules or sequences specified in the present application by stating a database code can be found under the database code L20916 in the NCBI database in the online version of 1 Apr. 2012.

However, the teaching of the invention can not only be performed by using the precise amino acid or nucleic acid sequences or applied to the precise amino acid or nucleic acid sequences of the biological macromolecules described herein, for example the alanine dehydrogenase described under database code L20916, but also using, or to, variants of such macromolecules which can be obtained by deletion, addition or substitution of one or more than one amino acid or nucleic acid. In a preferred embodiment, the expression “variant” of a nucleic acid sequence or amino acid sequence, hereinbelow used synonymously and exchangeably with the expression “homologue”, means, as used in the present context, a different nucleic acid or amino acid sequence which, in respect of the corresponding original wild-type nucleic acid sequence or wild-type amino acid sequence, has a homology, used herein synonymously with identity, of 70, 75, 80, 85, 90, 92, 94, 96, 98, 99% or more percent, where preferably amino acids other than those which form the catalytically active centre or other than those which are essential to the structure or folding are deleted or substituted, or the latter are merely conservatively substituted, for example a glutamate by an aspartate or a leucin by a valine. The prior art describes algorithms which can be used for calculating the extent of the homology of two sequences, for example Arthur Lesk (2008), Introduction to bioinformatics, 3^{rd} edition. In a further, more preferred embodiment of the present invention, the variant of an amino acid or nucleic acid sequence has, preferably in addition to the abovementioned sequence homology, essentially the same enzymatic activity of the wild-type molecule, or of the original molecule. For example, a variant of a polypeptide with the enzymatic activity of an alanine dehydrogenase has the same or essentially the same proteolytic activity, i.e. is capable of catalysing the conversion of pyruvate, ammonium and NAD(P)H, preferably NADPH, to alanine. In a particular embodiment, the expression “essentially the same enzymatic activity” means an activity in respect of the substrates of the wild-type polypeptide which is markedly above the background activity and/or differs by less than 3, more preferably 2, even more preferably one, order of magnitude from the K_{M} and/or k_{cat} values of the wild-type polypeptide in respect of the same substrates. In a further preferred embodiment, the expression “variant” of a nucleic acid or amino acid sequence comprises at least one active part or fragment of the nucleic acid or amino acid sequence, respectively. In a further preferred embodiment, the expression “active part”, as used in the present context, means an amino acid sequence or a nucleic acid sequence which is shorter than the full length of the amino acid sequence, or which encodes for a shorter length than the full length of the amino acid sequence, wherein the amino acid sequence or the encoded amino acid sequence with the shorter length than the wild-type amino acid sequence has essentially the same enzymatic activity as the wild-type polypeptide or a variant thereof. In a particular embodiment, the expression “variant” of a nucleic acid comprises a nucleic acid whose complementary strand will, preferably under stringent conditions, bind to the wild-type nucleic acid. The stringency of the hybridization reaction can be determined readily by a person skilled in the art and depends generally on the length of the probe, the temperatures during washing and the salt concentration. In general, longer probes require higher temperatures for the hybridization, whereas shorter probes work at low temperatures. Whether hybridization takes place will, in general, depend on the ability of the denatured DNA to anneal to complementary strands which are present in its environment, and to do so below the melting temperature. The stringency of hybridization reactions and corresponding conditions are described in greater detail in Ausubel et al. 1995. Information on identifying DNA sequences by means of hybridization can be found by a person skilled in the art in, inter alia, the text book “The DIG System Users Guide for Filter Hybridization” from Boehringer Mannheim GmbH (Mannheim, Germany, 1993) and in Liebl et al. (International Journal of Systematic Bacteriology 41: 255-260 (1991)). In a preferred embodiment, the hybridization takes place under stringent conditions, in other words only hybrids in which probe and target sequence, i.e. the polynucleotides treated with the probe, have at least 70% identity are generated. It is known that the stringency of the hybridization including the wash steps is influenced or determined by varying the buffer composition, the temperature and the salt concentration. In general, the hybridization reaction is carried out at relatively low stringency in comparison with the wash steps (Hybaid Hybridisation Guide, Hybaid Limited, Teddington, UK, 1996). For example, it is possible to employ, for the hybridization reaction, a buffer corresponding to 5×SSC buffer at a temperature of about 50° C.-68° C. Here, probes can also hybridize with polynucleotides that have less than 70% identity to the sequence of the probe. Such hybrids are less stable and are removed by washing under stringent conditions. Stringent washing conditions may be achieved, for example, by lowering the salt concentration to 2×SSC and optionally subsequently to 0.5×SSC (The DIG System User's Guide for Filter Hybridisation, Boehringer Mannheim, Mannheim, Germany, 1995), where a temperature of, in order of increasing preference, about 50° C.-68° C., about 52° C.-68° C., about 54° C.-68° C., about 56° C.-68° C., about 58° C.-68° C., about 60° C.-68° C., about 62° C.-68° C., about 64° C.-68° C., about 66° C.-68° C. is set. Temperature ranges of about 64° C.-68° C. or about 66° C.-68° C. are preferred. Optionally, it is possible to lower the salt concentration to correspond to a concentration of 0.2×SSC or 0.1×SSC. By increasing the hybridization temperature stepwise in steps of about 1-2° C. from 50° C. to 68° C., polynucleotide fragments may be isolated that, for example, in the order of increasing preference, at least 70% or at least 80% or at least 90%, at least 91%, at least 92%, at least 93%, at least 94%, at least 95%, at least 96%, at least 97%, at least 98% or at least 99% identity to the sequence of the nucleic acid molecule employed. Further instructions for hybridization are available on the market in the form of “kits” (e.g. DIG Easy Hyb from Roche Diagnostics GmbH, Mannheim, Germany, Catalogue No. 1603558). In a preferred embodiment, the expression “variant” of a nucleic acid, as used in the present context, comprises any nucleic acid sequence which codes for the same amino acid sequence as the original nucleic acid or for a variant of this amino acid sequence within the bounds of the degeneracy of the genetic code.

To carry out the teaching of the invention, it is essential that the Bacillus subtilis alanine dehydrogenase used does not comprise the wild-type sequence of the enzyme but a sequence in which Leu197 or an amino acid located at a homologous position in the amino acid sequence is exchanged for an amino acid with a positively charged side chain. In a preferred embodiment, the expression “homologous position” as used in the present context means that the corresponding position, when subjected to an alignment of the studied molecule, appears as a homologue to position X of the amino acid sequence of the Bacillus subtilis alanine dehydrogenase. A person skilled in the art is familiar with a large number of software packages and algorithms by means of which an alignment of amino acid sequences may be established. Examples of software packages processes comprise the ClustalW package provided by the EMBL (Larkin et al., 2007; Goujon et al. 2010) or are specified and described in Arthur M. Lesk (2008), Introduction to Bioinformatics, 3rd edition. In a preferred embodiment, the expression “amino acid” or “alanine” is understood as meaning a proteinogenic L-amino acid, or L-alanine, respectively, i.e. an amino acid used universally in nature by organisms for synthesizing polypeptides. The expression “amino acid” also comprises all crystal forms, salt forms or similar forms of the corresponding compound, as do all of the compounds mentioned in the present application. For example, histidine also comprises the salt of protonated histidine and a chloride ion.

It is essential for the activity of the alanine dehydrogenase that the starting materials of the catalysed reaction, in particular an inorganic nitrogen source, are present in a sufficient amount.

In a preferred embodiment, the expression “inorganic nitrogen source”, as used in the present context, is understood as meaning an inorganic nitrogen-containing salt which comprises ammonium or which can be metabolized to ammonium in the cell's metabolism. Examples comprise ammonium chloride, ammonium nitrate, ammonium sulphate, ammonium hydroxide, ammonium phosphate, ammonium carbonate and the like. In a preferred embodiment, the ammonium concentration in the medium amounts to from 0.05 to 5, more preferably from 0.1 to 3, most preferably from 0.5 to 3 g/l. The inorganic nitrogen source of the cell is preferably provided in accordance with the invention by adding a sufficient amount of the compound in question to the aqueous phase in which the reaction proceeds.

Amino acids with a positively charged side chains comprise the amino acids arginine, lysine and histidine, preferably arginine and lysine, most preferably arginine. Polypeptides according to the invention which are possible include, for example, the mutations Leu197Arg, Leu197Lys and Leu197His. In a preferred embodiment, the teaching of the invention expressly also comprises alanine dehydrogenases from organisms other than Bacillus subtilis as long as they are part of the group of the variants of the Bacillus subtilis alanine dehydrogenase.

In a further preferred embodiment, the teaching of the invention also comprises a polypeptide which, besides the abovementioned exchange at the position of Leu197, includes a further exchange at position Asp196 or at a position which is homologous thereto, this exchange being for an amino acid with a neutral or positively charged side chain. In a preferred embodiment, amino acids with a neutral side chain comprise the group comprising glycine, alanine, valine, leucine, tryptophan, tyrosine, isoleucine, serine, cysteine, threonine, glutamine, methionine, phenylalanine, proline and asparagine. In a preferred embodiment, amino acids with a positively charged side chain comprise the group comprising arginine, lysine and histidine, preferably arginine and lysine.

Nucleic acid molecules according to the invention comprise all unmodified or modified DNA and RNA molecules comprising a nucleic acid sequence coding for a polypeptide according to the invention. A person skilled in the art is familiar with standard methods of molecular biology or of synthetic chemistry, by means of which methods such nucleic acid molecules can be prepared, for example the polymerase chain reaction or solid-phase synthesis. In a preferred embodiment, the nucleic acid molecule is a nucleic acid molecule which comprises, or is, the nucleotide sequence shown in SEQ ID No. 2.

The teaching of the invention can be carried out not only using an isolated polypeptide, nucleic acid molecule or vector according to the invention, but also using a cell according to the invention as a whole-cell catalyst. In a preferred embodiment, the expression “whole-cell catalyst” as used in the present context is taken to mean an intact, viable and metabolically active cell which provides a desired enzymatic activity. The whole-cell catalyst can either transport the substrate to be metabolized, in the case of the present invention the alcohol or the oxidation product generated therefrom, into the interior of the cell, where it is metabolized by cytosolic enzymes, or else it may present the enzyme of interest on its surface, where it is exposed directly to substrates in the medium. A person skilled in the art is familiar with a large number of systems for preparing whole-cell catalysts, for example from DE 60216245. In an especially preferred embodiment, the cell expresses the polypeptide according to the invention with NADPH-dependent alanine dehydrogenase activity, the transaminase and/or the NADP⁺-dependent alcohol dehydrogenase intracellularly, i.e. the respective enzyme, after its expression, is permanently located in the interior of the cell, in particular in the cytosol of the cell or as a membrane protein or protein which is anchored in the membrane and which projects into the cytosol of the cell.

In a preferred embodiment, the cell used as the whole-cell catalyst, or the cell used as the expression system, is a prokaryotic cell, preferably a bacterial cell. In a further preferred embodiment, it is a mammalian cell. In a further preferred embodiment, it is a lower eukaryotic cell, preferably a yeast cell. Examples of prokaryotic cells comprise Escherichia, especially Escherichia coli, and strains of the genus Pseudomonas and Corynebacterium. Examples of lower eukaryotic cells comprise the genera Saccharomyces, Candida, Pichia, Yarrowia, Schizosaccharomyces, especially the strains Candida tropicalis, Schizosaccharomyces pombe, Pichia pastoris, Yarrowia lipolytica and Saccharomyces cerivisiae.

In the event that a whole-cell catalyst according to the invention is used, the entry of the molecule into the interior of the whole-cell catalyst may be limiting for the production of the desired substance in the case of some substrates, in particular those with long alkyl chains. In the case of long-chain alkanes and alcohols, it is preferred for the whole-cell catalyst to include an AlkL polypeptide. In a preferred embodiment, an “AlkL polypeptide” as used in the present context is a polypeptide which has at least 80, preferably 90, even more preferably 90% sequence identity to the Pseudomonas putida AlkL (database code CAB69081) over a length of 230 contiguous amino acids and one that preferably has the ability of promoting the import of long-chain alkanes into the interior of a cell. In a further embodiment, a “polypeptide of the AlkL family” as used in the present context is a polypeptide which is located in the outer membrane of a Gram-negative bacterium, which polypeptide includes the sequence motif DXWAPAXQ(V/A)GXR, where X represents a proteinogenic amino acid, and which polypeptide is preferably additionally Pseudomonas putida AlkL (database code CAB69081) or a variant thereof. Examples of members of the AlkL family comprise AlkL from Pseudomonas putida (database code CAB69081), Marinobacter aquaeolei VT8 (database code YP_{—}957722), Oceanicaulis alexandrii HTCC2633 (database code ZP_{—}00953584), Marinobacter manganoxydans MnI7-9 (database code ZP_{—}09158756), Caulobacter sp. K31 (database code YP_{—}001672217), Pseudomonas oleovorans (database code Q00595) and variants thereof.

The enzymatically active polypeptides employed in accordance with the invention, in particular alcohol dehydrogenase, transaminase and alanine dehydrogenase, may, however, also be in each case a preparation of the polypeptide according to the invention in any stage of purification, from the crude lysate to the isolated polypeptide. The cell may comprise one or more than one nucleic acid sequence coding for an enzyme used according to the invention, either on a plasmid or integrated into the cell's genome. A person skilled in the art is familiar with a large number of processes by means of which enzymatically active polypeptides can be overexpressed in suitable cells and purified or isolated. Thus, any of the expression systems available to a person skilled in the art may be used for expressing the polypeptides, for example vectors of the pET or pGEX type. Methods which are suitable for the purification are chromatographic methods, for example the purification by affinity chromatography of a tagged recombinant protein using an immobilized ligand, for example a nickel ion in the case of a histidine tag, of immobilized glutathione in the case of a glutathione S-transferase fused to the target protein, or immobilized maltose in the case of a tag comprising maltose-binding protein.

The use of the polypeptide according to the invention in isolated form is expressly recommended for a series of applications. In a preferred embodiment, the expression “isolated” as used in the present context means that the enzyme is present in a more pure and/or in a concentrated form than in its natural source. In a preferred embodiment, the enzyme is considered to be isolated when it is a polypeptide enzyme and when it amounts to more than 60, 70, 80, 90 or preferably 95% of the protein content, by mass, of the preparation in question. A person skilled in the art is familiar with a large number of methods for measuring the mass of a protein in a solution, for example the visual estimate with reference to the thickness of corresponding protein bands on SDS polyacrylamide gels, NMR spectroscopy or methods based on mass spectrometry.

The purified enzymatically active polypeptides can be employed either in soluble form or in immobilized form. A person skilled in the art is familiar with suitable methods by means of which polypeptides may be immobilized covalently or noncovalently on organic or inorganic solid phases, for example by sulphhydryl coupling chemistry (for example kits from Pierce).

The enzymes used in accordance with the invention are preferably recombinant enzymes. In a preferred embodiment, the expression “recombinant” as used in the present context is understood as meaning that the nucleic acid molecule in question does not occur naturally and/or that it has been generated using recombinant methods. In a preferred embodiment, a recombinant protein is considered one where the polypeptide in question is encoded by a recombinant nucleic acid. In a preferred embodiment, a recombinant cell as used in the present context is understood as meaning a cell which includes at least one recombinant nucleic acid or one recombinant polypeptide. A person skilled in the art is familiar with methods which are suitable for generating recombinant molecules or cells, for example those described in Sambrook/Fritsch/Maniatis (1989): Molecular Cloning: A Laboratory Manual, Cold Spring Harbor Laboratory Press, 2^{nd} edition.

A prerequisite for using the teaching of the invention is the presence of an aqueous phase, i.e. an aqueous culture medium or reaction medium which is suitable for the at least temporary maintenance or culturing of the cell or for the activity of the polypeptide according to the invention. A person skilled in the art is familiar with a large number of aqueous culture media which are suitable for the maintenance or the culturing of cells, in particular biotechnologically important cells. These include not only complete media such as an LB medium, but also minimal media such as M9 media and selective media, for example those which have a high salt concentration and which therefore allow only the growth of halophilic or at least salt-tolerant organisms. In a preferred embodiment, the expression “aqueous phase” as used in the present context is understood as meaning a water-based reaction medium or culture medium which is essentially immiscible with hydrophobic solvents and which, in respect of all relevant factors, in particular pH, salt content and temperature, is such that it at least temporarily maintains or promotes the viability of cells, preferably microorganisms, which are present therein. The temperature requirements of various biotechnologically important cells can be found in text books of microbiology and molecular biology, for example Fuchs/Schlegl, 2008. In a preferred embodiment, the pH of the aqueous culture medium is between 4 and 9, more preferably between 4.5 and 8.5, most preferably between 6.5 and 7.5, at the time when contact is made. In a further preferred embodiment, the temperature is between 5 and 42° C., more preferably between 15 and 40° C., most preferably between 20 and 37° C.

The use of enzymes requires the presence of all the substrates which are necessary. For example, the activity of alanine dehydrogenase requires the presence of the substrates ammonium, a suitable reduced redox factor and pyruvate. Furthermore, the activity of enzymes requires a suitable aqueous solution, i.e. a solution which, in respect of the required buffer present, the pH, the temperature, the salt concentration, the presence of required cofactors or activity-promoting or activity-maintaining further polypeptides and other relevant factors, is suitable for at least temporarily maintaining the activity of the enzyme. Methods for choosing suitable solutions and enzymatic tests by means of which the activity of enzymes can be determined are known to a person skilled in the art and described in the prior art, for example in Cornish-Bowden (1995), Fundamentals of Enzyme Kinetics, Portland Press Limited.

According to the invention, the cell according to the invention expresses not only the modified Bacillus subtilis alanine dehydrogenase, but, in a preferred embodiment, also an NADP⁺-dependent alcohol dehydrogenase. In a preferred embodiment, the expression “alcohol dehydrogenase” as used in the present context is understood as meaning an enzyme which oxidizes an aldehyde or ketone to give the corresponding primary or secondary, or other, alcohol. Examples comprise the NADP⁺-dependent alcohol dehydrogenases from E. coli (YjgB, database code ZP_{—}07117674, and a YahK, database code BAE76108.1) and the alcohol dehydrogenase from Ralstonia sp. (SEQ ID No. 5) and the respective variants thereof. In an especially preferred embodiment, it is the alcohol dehydrogenase from Ralstonia sp. (SEQ ID No. 5).

According to the invention, the cell according to the invention expresses not only the modified Bacillus subtilis alanine dehydrogenase and/or the NADP⁺-dependent alcohol dehydrogenase, but also, in a preferred embodiment, a transaminase. In a preferred embodiment, the expression “transaminase” as used in the present context is understood as meaning an enzyme which catalyses the transfer of amino groups from a donor molecule, preferably an amino acid, to an acceptor molecule, preferably a ketocarboxylic acid. In an especially preferred embodiment, the transaminase is selected from the group which comprises the ω-transaminase from Chromobacterium violaceum (database code NP_{—}901695), Pseudomonas putida (database code YP_{—}001668026), Pseudomonas putida (database code YP_{—}001668026.1 or YP_{—}001671460); Rhodobacter sphaeroides (strain ATCC 17023; database code YP_{—}353455) and variants thereof, and it is preferably the transaminase from Chromobacterium violaceum (database code NP_{—}901695).

In the event that at least one whole-cell catalyst is used, and if the reaction time is prolonged, it should be taken into consideration that the conditions are compatible with the viability of the at least one cell employed as the whole-cell catalyst. A person skilled in the art can find conditions and solutions which make possible the maintenance of such cells in a viable state in standard works, for example Fuchs/Schlegel (2007) Allgemeine Mikrobiologie, 2008, Georg Thieme Verlag.

The method according to the invention can be applied to a large number of industrially relevant alcohols. In a preferred embodiment, they take the form of a ω-hydroxycarboxylic acid or an ester, preferably methyl ester, thereof, which is oxidized and aminated to give an ω-aminocarboxylic acid. In a further embodiment, it is a diol, which is oxidized and aminated to give a diamine. In a further preferred embodiment, the primary alcohol is a hydroxyalkylamine. The length of the carbon chain varies in this context, and x amounts to at least 3. Preferably, the carbon chain has more than three C atoms, i.e. x=4, 5, 6, 7, 8, 9, 10, 11, 12, 13, 14, 15, 16, 17, 18, 19, 20 or more. Examples of compounds comprise ω-hydroxylauric acid, methyl ω-hydroxy-laurate, and alkane diols, in particular 1,8-octane diol and 1,10-decane diol.

Others which are suitable are α-hydroxycarboxylic acids, preferably those which can be oxidized to give the α-ketocarboxylic acids, i.e. those of the formula R^{S}—C(OH)H—COOH, which, in turn, can be converted by amination into the proteinogenic amino acids, among which in particular are essential amino acids such as methionine and lysine. Specific examples comprise those acids in which R^{S} is a substituent from the group comprising H, methyl, —(CH₂)₄—NH₂, —(CH₂)₃—NH—NH—NH₂, —CH₂—CH₂—S—CH₃, —CH(CH₃)₂, —CH₂—CH(CH₃)₂, —CH₂-(1H-indol-3-yl), —CH(OH)—CH₃, —CH₂-phenyl, —CH(CH₃)—CH₂—CH₃. Other secondary alcohols comprise 2-alkanols, for example 2-propanol, 2-butanol, 2-pentanol, 2-hexanol and the like. Others which are suitable are secondary polyhydric alcohols, for example alkanediols such as ethanediol, alkanetriols such as glycerol and pentaerythritol. Other examples comprise cycloalkanols, preferably cyclohexanol and bis(p-hydroxycyclohexyl)methane, the alcohols from the group H₃C—C(OH)H—(CH₂)ₓ—R⁴, where R⁴ is selected from the group comprising —OH, —SH, —NH₂ and —COOR⁵, x being at least 3, and R⁵ is selected from the group which comprises H, alkyl and aryl.

In the case of alcohols of the formula alcohols of H₃C—C(OH)H—(CH₂)ₓ—R⁴ the length of the carbon chain varies, and x amounts to at least 3. Preferably, the carbon chain includes more than three carbon atoms, i.e. x=4, 5, 6, 7, 8, 9, 10, 11, 12, 13, 14, 15, 16, 17, 18, 19, 20 or more. A large number of secondary alcohols are commercially available and can be employed directly in commercially available form. Alternatively, the secondary alcohol may be generated biotechnologically, either beforehand or in situ, for example by hydroxylating an alkane by suitable alkane oxidases, preferably monooxygenases.

In the case of secondary alcohols of formula H₃C—C(OH)H—(CH₂)ₓ—R⁴, R⁴, in an especially preferred embodiment, is selected from the group comprising —OH and —COOR⁵, x being at least 11, and R⁵ is selected from the group comprising H, methyl, ethyl and propyl.

The primary or secondary alcohol is especially preferably a sugar alcohol which, as used in a preferred embodiment, such as herein, is understood as meaning a carbohydrate which includes at least one hydroxyl group. In an especially preferred embodiment, it is a dicyclic sugar alcohol. In a preferred embodiment, such as herein, a “dicyclic sugar alcohol” is understood as meaning a sugar alcohol which is capable of forming two ring systems, at least temporarily. In a most preferred embodiment, a sugar alcohol is a dianhydrohexitol or a compound from the group comprising 1,4:3,6-dianhydro-D-mannitol, 1,4:3,6-dianhydro-D-glucitol (isosorbide) and 1,4:3,6-dianhydro-L-iditol.

If the alcohols to be converted in accordance with the invention are not available as such, but only in the form of alkane precursors, there is the option that the cell according to the invention additionally expresses a monooxygenase or to extend the method according to the invention by a step in which a monooxygenase hydroxylates an alkane. In an especially preferred embodiment, the monooxygenase is a monooxygenase from the AlkB family. AlkB is an oxidoreductase from the Pseudomonas putida AlkBGT system and is known for its hydroxylase activity. The latter is dependent on two further polypeptides, AlkG and AlkT. AlkT is characterized as an FAD-dependent rubredoxin reductase which transfers electrons from NADH to AlkG. AlkG is a rubredoxin, a ferruginous redox protein which acts as a direct electron donor for AlkB. In a preferred embodiment, the expression “monooxygenase from the alkB family” as used in the present context is understood as meaning a membrane-associated alkanehydroxylase. In a further preferred embodiment, the same expression “alkanehydroxylase of the alkB type” is understood as meaning a polypeptide with a sequence homology of, with increasing preference, at least 75, 80, 85, 90, 92, 94, 96, 98 or 99% to the sequence of the AlkB from Pseudomonas putida Gpo1 (database code: CAB54050.1, this database code and all other database codes used in the present document are from the Genbank protein database of the NCBI in the release available from 9 Nov. 2011). The expression “sequence” as used in the present context may refer to the amino acid sequence of a polypeptide and/or to the nucleic acid sequence encoding it.

In a further preferred embodiment, the monooxygenase is cytochrome P450 monooxygenase of the CYP153 family. In a preferred embodiment, the expression “cytochrome P450 monooxygenase of the CYP153 family” is understood as meaning a cytosolic oxidase which is part of a 3-component system which furthermore comprises a ferredoxin and a ferredoxin reductase, which cytosolic oxidase has an alkane binding site and is capable of hydroxylating alkanes. In an especially preferred embodiment, it is an enzyme which has at least 80, preferably 90, most preferably 95 or 99 percent sequence identity to the cytochrome P450 monooxygenase of the CYP153 family from Alcanivorax borkumensis SK2 (database code YP_{—}691921) or an enzyme which comprises a polypeptide sequence which has at least 80, preferably 90, most preferably 95 or 99 percent sequence identity to the cytochrome P450 monooxygenase from the CYP153 family from Alcanivorax borkumensis SK2 (database code YP_{—}691921) and which additionally has alkane hydroxylase activity. In a preferred embodiment, the expression “alkane hydroxylase activity” as used in the present context is understood as meaning the ability of catalysing the hydroxylation of alkanes or unsubstituted linear alkyl radicals comprising at least five, preferably twelve, carbon substance radicals. In a further preferred embodiment, the expression “cytochrome P450 monooxygenase of the CYP153 family” is understood as meaning a non-membrane-bound oxidase which comprises a binding site for alkanes, unsubstituted linear alkyl radicals comprising at least five, preferably twelve, carbon substance radicals or monohydroxylated alkanes and whose polypeptide chain comprises the motif LL(I/L)(V/I)GGNDTTRN. In a preferred embodiment, a “cytochrome P450 monooxygenase of the CYP153 family” as used in the present context is a cytochrome P450 monooxygenase from the CYP153 family from Alcanivorax borkumensis SK2 (database code YP_{—}691921) or a variant thereof, which preferably has alkane hydroxylase activity.

The present application comprises a sequence listing with the following polypeptide (Polyp) sequences and nucleotide (DNA) sequences:

| SEQ | | |
|---|---|---|
| ID No. | Type | Description |
| 1 | Polyp | Alanine dehydrogenase from Bacillus subtilis with |
| | | Leu197Arg and Asp196Ala |
| 2 | DNA | Nucleotide sequence coding for alanine dehydrogenase of |
| | | Bacillus subtilis with Leu197Arg and Asp196Ala |
| 3 | Polyp | Alcohol dehydrogenases from E. coli (YjgB, database |
| | | code ZP_07117674) |
| 4 | Polyp | Alcohol dehydrogenases from E. coli (YahK, database |
| | | code BAE76108.1) |
| 5 | Polyp | Alcohol dehydrogenase from Ralstonia sp. (previously |
| | | SEQ ID 10) |
| 6 | Polyp | Transaminase from Chromobacterium violaceum (database |
| | | code NP_901695) |
| 7 | Polyp | Amino transferase from Pseudomonas putida (database |
| | | code YP_001668026) |
| 8 | Polyp | Amino transferase from Pseudomonas putida (database |
| | | code YP_001668026.1) |
| 9 | Polyp | Amino transferase from Pseudomonas putida (database |
| | | code YP_001671460) |
| 10 | Polyp | Amino transferase from Rhodobacter sphaeroides (strain |
| | | ATCC 17023; database code YP_353455) |
| 11 | DNA | Oligodeoxy nucleotide AlaDHfw (previously Seq ID 5) |
| 12 | DNA | Oligodeoxy nucleotide AlaDHrv (previously 6) |
| 13 | DNA | Oligodeoxy nucleotide AlaDH_D196A/L197Rfw |
| | | (previously 8) |
| 14 | DNA | Oligodeoxy nucleotide AlaDH_D196A/L197Rrv |
| | | (previously 9) |
| 15 | DNA | Oligodeoxy nucleotide ADHfw (previously 11) |
| 16 | DNA | Oligodeoxy nucleotide ADHrv (previously 12) |
| 17 | Polyp | Amino transferase from Paracoccus denitrificans |
| | | (previously 14) |
| 18 | DNA | Oligodeoxy nucleotide pCR6fw (previously 15) |
| 19 | DNA | Oligodeoxy nucleotide pCR6rv (previously 16) |
| 20 | DNA | Oligodeoxy nucleotide pCR6_L417Mfw (previously 18) |
| 21 | DNA | Oligodeoxy nucleotide pCR6_L417Mrv (previously 19) |
| 22 | Polyp | Alanine dehydrogenase from Bacillus subtilis with |
| | | Leu197Arg and Asp196Ala with His-Tag |
| 23 | DNA | Nucleotide sequence coding for alanine dehydrogenase |
| | | from Bacillus subtilis with Leu197Arg and Asp196Ala |
| | | with His-Tag |

The features of the invention disclosed in the preceding description, the claims and the drawings can be essential to realizing the invention in its various embodiments, either individually or else in any combination.

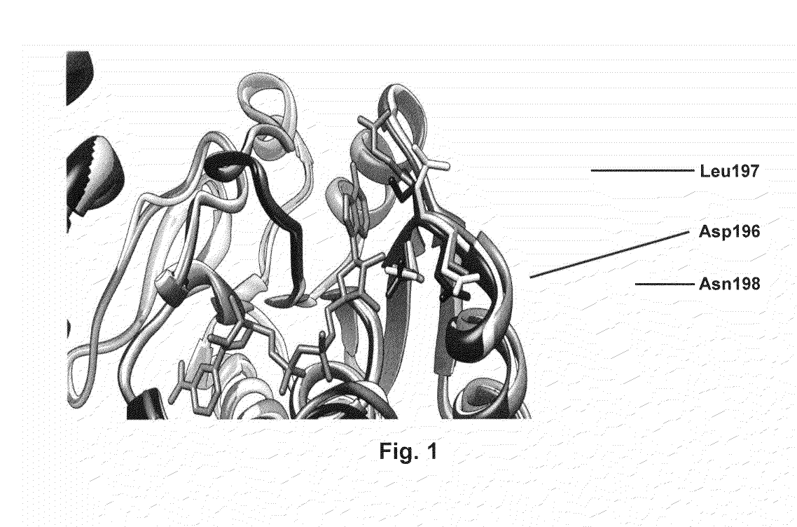

FIG. 1 shows the structural superimposition of the homology model of the AlaDH from Bacillus subtilis (white; SEQ ID No. 1), of the homology model of the AlaDH from Shewanella sp. (grey) and of the crystal structure of the AlaDH from Mycobacterium tuberculosis (black; PDB code: 2VHW). The amino acid Asp196, which is shown, of the BasAlaDH corresponds to the amino acid Asp198 in the SheAlaDH and to the amino acid Asp198 in the MtAlaDH, respectively. The amino acid Leu197 of the BasAlaDH corresponds to the amino acid Arg199 in the SheAlaDH and to the amino acid Ile199 in the MtAlaDH, respectively. The amino acid Asn198 of the BasAlaDH corresponds to the amino acid Ser200 in the SheAlaDH and to the amino acid Asn200 of the MtAlaDH, respectively.

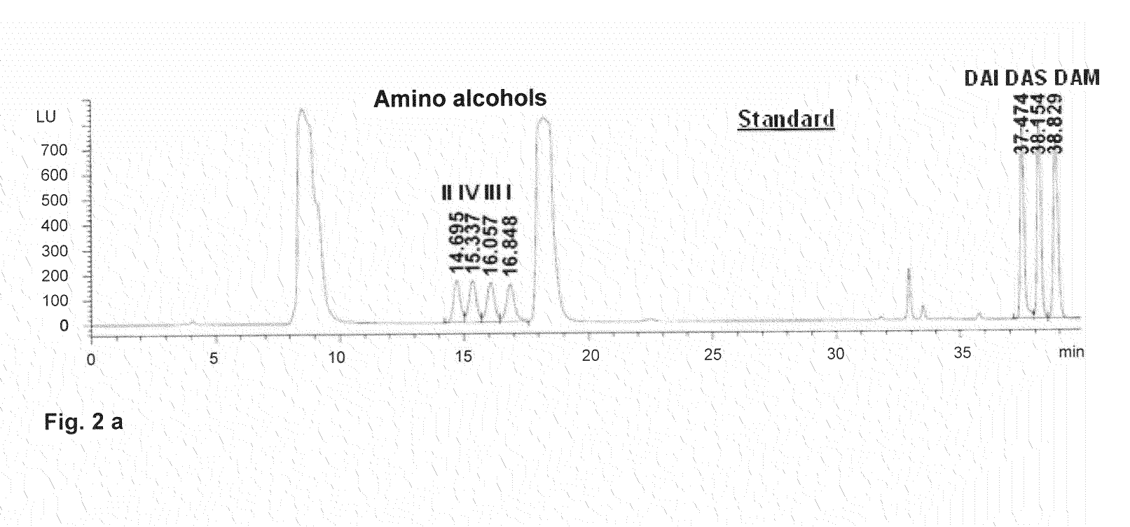

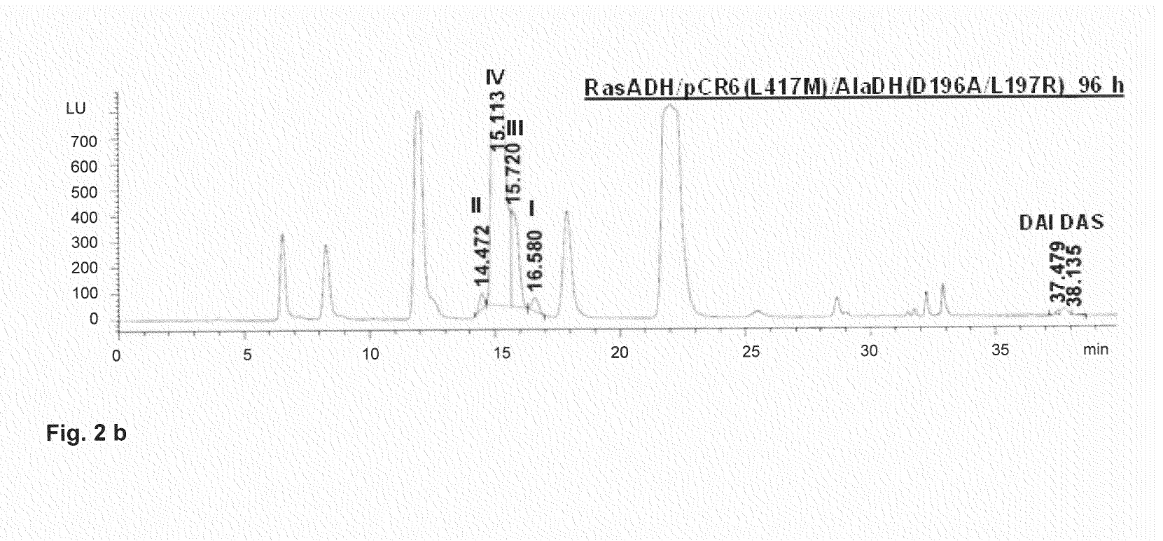

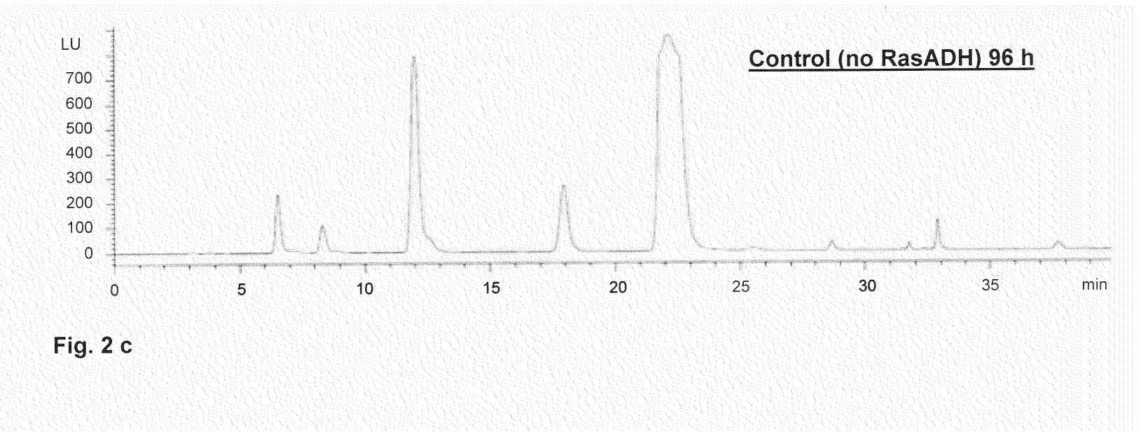

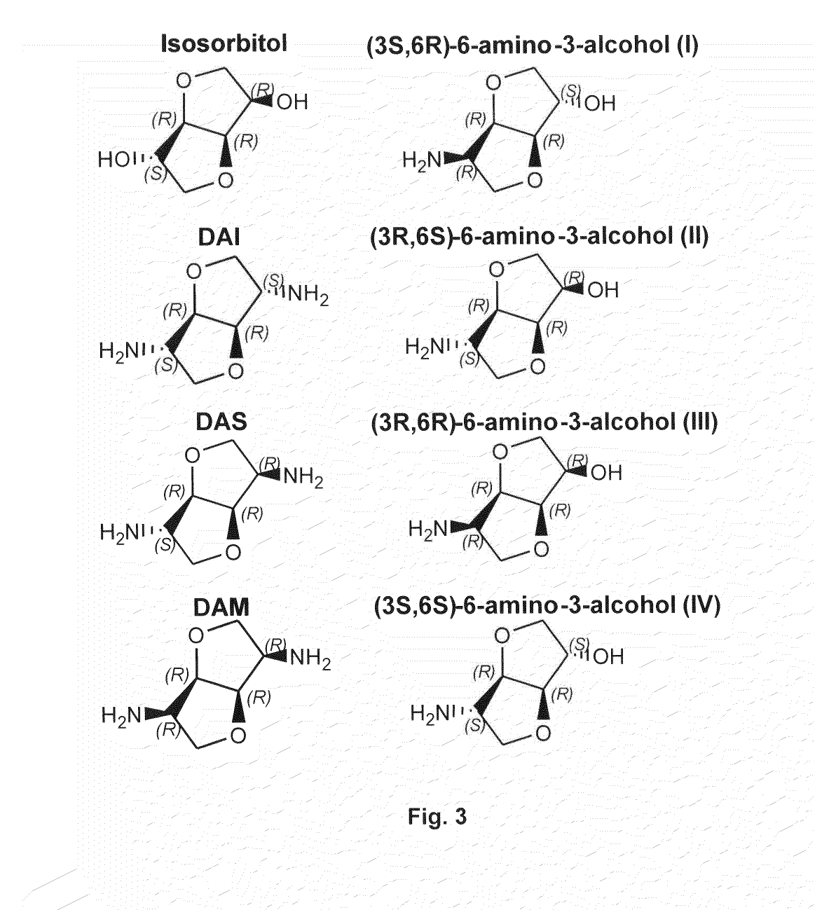

FIG. 2 shows the FMOC/HPLC analysis of the reaction of isosorbitol and ammonium salt after 96 hours, as catalysed by the three enzymes RasADH, pCR6(L417M) and AlaDH(D196A/L197R). The following are shown: (a) the standards (in each case 1 mM of the amino alcohols I, II, III and IV as shown in FIG. 3 + in each case 1 mM of the diamines DAI, DAS and DAM), (b) the reaction as catalysed by RasADH, pCR6(L417M) and AlaDH(D196A/L197R) after 96 h, (c) the control reaction as in (b), but without RasADH, after 96 h. For the derivatization, 20 μl of the respective reaction sample were transferred into an HPLC vial containing 60 μl of 0.5 M sodium borate pH 9.0, the sample was mixed thoroughly, and 80 μl of FMOC reagent (Alltech Grom) were added. Excess FMOC reagent was captured by the addition of 100 μl of EVA reagent (Alltech Grom). The conditions for the HPLC analysis were established by adding 440 μl of 50 mM sodium acetate, pH 4.2+70% acetonitrile (v/v). Chromatographic conditions: Agilent SB-C8 column (4.6×150 mm); flow rate: 1 ml/min; injection volume: 20 μl; buffer A: 50 mM Na acetate pH 4.2+20% acetonitrile (v/v); buffer B: 50 mM Na acetate pH 4.2+95% acetonitrile (v/v); gradient: 0 min 16% B, 5 min 16% B, 25 min 18% B, 28 min 52% B, 40 min 25% B.

FIG. 3 shows the chemical formulae of the starting substrate isosorbitol (1,4:3,6-dianhydro-D-sorbitol), of the stereoisomers of the amino alcohol (I to IV) and of the stereoisomeric forms of the diamine end product (DAI: 2,5-diamino-1,4:3,6-dianhydro-2,5-dideoxy-L-iditol, DAS: 2,5-diamino-1,4:3,6-dianhydro-2,5-dideoxy-D-sorbitol and DAM: 2,5-diamino-1,4:3,6-dianhydro-2,5-dideoxy-D-mannitol).

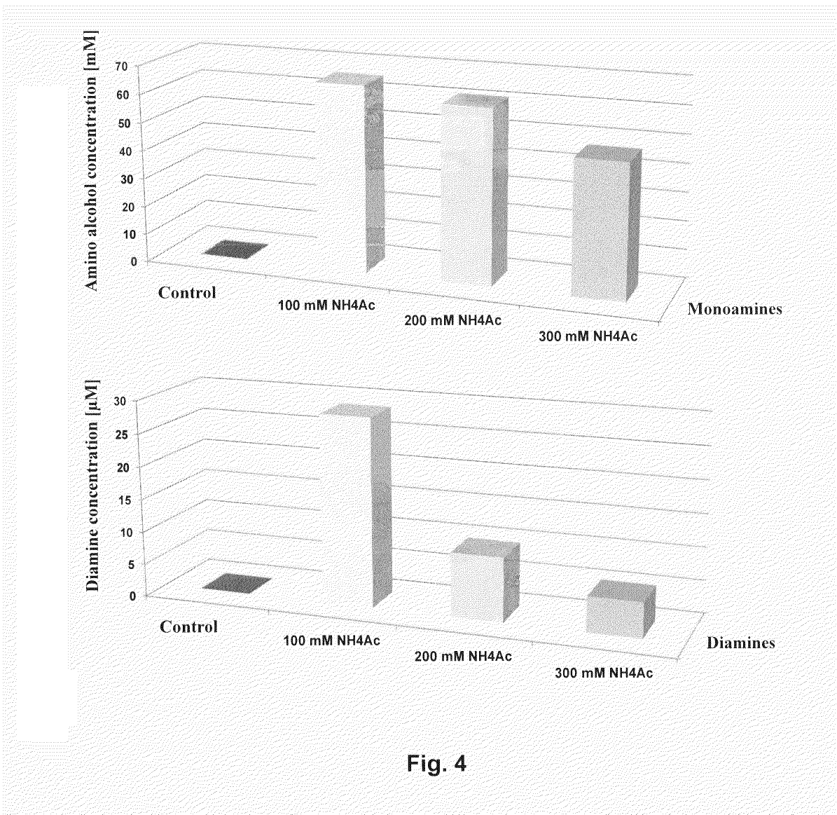

FIG. 4 shows the monoamine and diamine yields from the FMOC/HPLC analysis of the reaction of isosorbitol and ammonium acetate at different ammonium concentrations, as catalysed by RasADH, pCR6(L417M) and AlaDH(D196A/L197R). Reaction conditions: 300 mM isosorbitol, 2 mM NADP⁺, 100-300 mM NH₄OAc, 5 mM L-alanine, 0.3 mM PLP, 132 μM RasADH, 40 μM pCR6(L417M), 24 μM AlaDH(D196A/L197R) in 25 mM Hepes/NaOH, pH 8.3; incubation at 30° C.

### EXAMPLE 1

### Modifying the Cosubstrate Specificity of an Alanine Dehydrogenase Using Bioinformatics Analysis and Protein Engineering

Structural homologues of the L-alanine dehydrogenase (AlaDH) from Bacillus subtilis (BasAlaDH; SEQ ID No. 1) were identified with the aid of the server HHpred (Bioinformatics Toolkit; MPI Tübingen). A global multiple sequence alignment (MSA) was generated using HHblits (Bioinformatics Toolkit; MPI Tübingen; URL: http://toolkit.tuebingen.mpg.de/hhpred) in three iterations and with activated “Secondary Structure Score”. The most similar protein (51% sequence identity) proved to be the AlaDH from Mycobacterium tuberculosis (MtAlaDH). The crystal structures of this AlaDH (PDB codes: 2VHW, 2VHX, 2VHY and 2VHZ) were used as a structural template for generating homology models of the BasAlaDH and the AlaDH from Shewanella sp. (SheAlaDH) by means of the HHpred server (FIG. 1).

The MtAlaDH, like the enzyme from Bacillus subtilis, has a redox factor specificity for NADH, whereas the SheAlaDH, as the only known AlaDH, is capable of enzymatically converting both NADH and NADPH (Ashida et al. (2004) J. Mol. Catal. B: Enzym. 30, 173-176). A structural superimposition of the homology models of BasAlaDH and SheAlaDH with the MtAlaDH reference structure was calculated using the programme Chimera (Version 1.5.3; URL: http://www.cgl.ucsf.edu/chimera/ with the function “Matchmaker”; URL http://www.cgl.ucsf.edu/chimera/docs/ContributedSoftware/matchmaker/matchmaker.html) (FIG. 1). The Needleman-Wunsch algorithm using the Blossum-62 template was used for this purpose, and the “Secondary Structure Score” was set to 30%. The iteration termination used was a distance of 1.0 Å between pair-wise atoms.

The structural superimposition of the three AlaDHs mentioned shows a conserved aspartate residue in the vicinity of the “Rossmann fold” motif, which residue is obviously responsible for the NADH specificity (Asp196 in SEQ ID No. 1; see FIG. 1).

The conserved aspartate residue was the starting point for constructing the BasAlaDH enzyme variants D196A/L197R, with the SheAlaDH structure being used as template. As demonstrated by the structural superimposition (FIG. 1), the D196A substitution prevents formation of hydrogen bridges from Asp196 to the 2′-OH and the 3′-OH of the NADH ribose, but makes possible the binding of the NADPH phosphate, located at the 2′-OH of the ribose, to the enzyme. As a result of the substitution L197R, the new cosubstrate specificity is favoured further as the result of the formation of salt bridges between the introduced arginine residue and the NADPH phosphate group.

The gene of the Bacillus subtilis AlaDH (SEQ ID No. 4) was amplified from the plasmid pUC18-AlaDH by polymerase chain reaction (PCR) using the oligodeoxy nucleotides AlaDHfw (SEQ ID No. 11) and AlaDHrv (SEQ ID No. 12), cleaved at the 3′ end using the restriction enzyme KpnI and ligated with the expression vector pASK-IBA35(+) (IBA GmbH, Göttingen) which had been cleaved with the restriction enzymes EheI and KpnI. The resulting expression plasmid pASK-IBA35(+)-AlaDH on which the BasAlaDH with an N-terminal His₆-tag is encoded was verified by means of analytical restriction digestion and DNA sequencing. The plasmid which codes for the enzyme variant D196A/L197R was generated by site-directed mutagenesis using the oligodeoxy nucleotides AlaDH_D196A/L197Rfw (SEQ ID No. 13) and AlaDH_D196A/L197Rrv (SEQ ID No. 14), using the QuikChange method. The resulting expression plasmid pASK-IBA35(+)-AlaDH(D196A/L197R) was verified by means of DNA sequencing.

The expression plasmids for the BasAlaDH and its variant D196A/L197R were subsequently used for transforming E. coli BL21 (Sambrook et al. (2001) Molecular cloning: a laboratory manual, 3rd edition. Cold Spring Harbor Laboratory Press). Gene expression was induced in each case in LB medium (Sambrook et al. 2001) supplemented with 100 μg/ml ampicillin (2 L culture volume in a 5-l shake flask) at 30° C. in the exponential growth phase at OD₅₅₀=0.5 by addition of 0.2 μg/ml anhydrotetracyclin (aTc; Acros, Geel, Belgium). After an induction time of 3 hours, the culture was harvested, and the cells were taken up in 40 mM Hepes/NaOH pH 7.5, 0.5 M NaCl and disrupted mechanically in a French press homogenizer (G. Heinemann, Schwäbisch Gmünd). The clear supernatant was applied to a Chelating Sepharose™ Fast Flow column loaded with Zn²⁺ (bed volume 2.8 ml; GE Healthcare, Munich), and the enzymes with His₆-tag fusion were eluted using a linear imidazole/HCl concentration gradient of from 0 to 500 mM in 40 mM Hepes/NaOH pH 7.5, 0.5 M NaCl. The elution fractions were concentrated by ultrafiltration and purified chromatographically by means of gel filtration on Superdex200 (GE Healthcare) in the presence of 25 mM Hepes/NaOH pH 8.3.

The Michaelis constant (K_{M}) and the turnover number (k_{cat}) of the two purified enzymes for the cosubstrates NADH and NADPH were determined with the aid of reductive amination of pyruvate, and the catalytic efficiency (k_{cat}/K_{M}) was calculated (Table 1). The enzyme assays were composed as follows:

| Reagent or enzyme | Final concentration in the mixture | |
|---|---|---|
| Pyruvate | 5 | mM |
| Ammonium acetate | 200 | mM |
| NADH/H⁺ or NADPH/H⁺ | 10-500 | μM |
| BasAlaDH (wild type or | 0.5 | μM |
| double mutant D196A/L197R) | | |
| Hepes/NaOH buffer pH 8.3 | 25 | μM |
| Total volume | 250 | μl |

Here, it was found that the substitutions D196A and L197R successfully modified the cosubstrate specificity of the BasAlaDH from NADH to NADPH, while the catalytic efficiency in respect of the reductive amination of pyruvate was virtually unchanged.

TABLE 1
K_{M} and k_{cat} values for the BasAlaDH-catalysed reductive amination of pyruvate
using NADH or NADPH

| | NADH | | | NADPH | | |
|---|---|---|---|---|---|---|
| | K_{M} | k_{cat} | k_{cat}/K_{M} | K_{M} | k_{cat} | k_{cat}/K_{M} |
| Enzyme | [μM] | [min⁻¹] | [μM⁻¹/min⁻¹] | [μM] | [min⁻¹] | [μM⁻¹/min⁻¹] |
| Wild type AlaDH | 14 ± 2 | 838 ± 21 | 59.9 | 448 ± 135 | 666 ± 114 | 1.5 |
| Mutant D196A/L197R | 887 ± 360 | 2669 ± 719 | 3.0 | 32 ± 3 | 1730 ± 39 | 54.1 |

### EXAMPLE 2

### Synthesis of Monoamines and Diamines from Isosorbitol and Ammonium Salts by the Coupled Enzymatic Reaction of an Alcohol Dehydrogenase, an Amino Transferase and an Alanine Dehydrogenase

The structural gene of the alcohol dehydrogenase from Ralstonia sp. (SEQ ID No. 5) was amplified from the plasmid pEam-RasADH (Lavandera et al. (2008) J. Org. Chem. 73, 6003-6005) by means of PCR using the oligodeoxy nucleotides ADHfw (SEQ ID No. 15) and ADHrv (SEQ ID No. 16), cleaved at the 3′ end using the restriction enzyme KpnI and finally ligated with the expression vector pASK-IBA35(+) which had been cleaved with the restriction enzymes EheI and KpnI. The resulting expression plasmid pASK-IBA35(+)-RasADH, on which the alcohol dehydrogenase with an N-terminal His₆-tag is encoded, was verified by analytical restriction digestion and DNA sequencing.

The gene of the amino transferase from Paracoccus denitrificans (SEQ ID No. 17) was amplified from the plasmid pET21a(+)-pCR6 by means of PCR using the oligodeoxy nucleotides pCR6fw (SEQ ID No. 18) and pCR6rv (SEQ ID No. 19), cleaved at the 3′ end using the restriction enzyme HindIII and finally ligated with the expression vector pASK-IBA35(+), which had been cleaved with the restriction enzymes EheI and HindIII. The resulting expression plasmid pASK-IBA35(+)-pCR6, on which the amino transferase with an N-terminal His₆-tag is encoded, was verified by analytical restriction digestion and DNA sequencing. The plasmid which encodes for the enzyme variant L417M of the amino transferase was generated by site-directed mutagenesis of the plasmid pASK-IBA35(+)-pCR6 using the QuikChange method (Agilent, Waldbronn) using the oligodeoxy nucleotides pCR6_L417Mfw (SEQ ID No. 20) and pCR6_L417Mrv (SEQ ID No. 21). The resulting expression plasmid pASK-IBA35(+)-pCR6(L417M) was verified by means of DNA sequencing.

The expression plasmid used for the D196A/L197R mutant of the AlaDH from Bacillus subtilis (SEQ ID No. 1) was pASK-IBA35(+)-AlaDH(D196A/L197R) from Example 1.

Each of the expression plasmids pASK-IBA35(+)-RasADH, pASK-IBA35(+)-pCR6(L417M) and pASK-IBA35(+)-AlaDH(D196A/L197R) for the three enzymes were then used for the transformation of E. coli BL21. The gene expression in the three resulting strains was induced in each case in LB medium with 100 □g/ml ampicillin (2 l culture volume in a 5-l shake flask) at 30° C. in the exponential growth phase at OD₅₅₀=0.5 by adding 0.2 μg/ml aTc. After an induction time of 3 hours, the culture was harvested, and the cells were taken up in 40 mM Hepes/NaOH pH 7.5, 0.5 M NaCl and disrupted mechanically in a French press homogenizer. The clear supernatant was applied to a Chelating Sepharose™ Fast Flow column loaded with Zn²⁺, and the enzymes with His₆-tag fusion were eluted using a linear imidazole/HCl concentration gradient of from 0 to 500 mM in 40 mM Hepes/NaOH pH 7.5, 0.5 M NaCl. The elution fractions were concentrated by ultrafiltration and purified chromatographically by means of gel filtration on Superdex200 in the presence of 25 mM Hepes/NaOH pH 8.3.

The three purified enzymes were employed directly for the amination of isosorbitol (1,4:3,6-dianhydro-D-sorbitol) with recycling of the redox factors NADP⁺ and L-alanine. The enzyme assay was composed as follows:

| Reagent or enzyme | Final concentration in the mixture | |
|---|---|---|
| Hepes/NaOH buffer pH 8.3 | 25 | mM |
| Isosorbitol | 300 | mM |
| NADP⁺ | 2 | mM |
| L-Alanine | 5 | mM |
| Pyridoxal phosphate (PLP) | 0.3 | mM |
| Ammonium acetate (NH₄OAc) | 100-300 | mM |
| Alcohol dehydrogenase | 132 | μM |
| Amino transferase (L417M) | 40 | μM |
| Alanine dehydrogenase (D196A/L197R) | 24 | μM |
| Total volume | 250 | μl |

After incubation for a period of from 0 to 96 h at 30° C., the formation of monoamines and diamines as the reaction products was detected and quantitatively determined by adding an excess of FMOC reagent (Alitech Grom, Rottenburg-Hailfingen; see FIG. 1) by means of HPLC (Agilent 1200 series; see FIG. 2) with the aid of a fluorescence detector.

FIG. 2 (centre) shows the chromatogram of the resolution of the reaction mixture. At retention times of 15.113 min, 15.720 min, 16.580 and 14.472 min, product peaks were observed which, according to the standards included, could be assigned to the 6-amino-3-alcohol in the 4 possible stereoisomeric forms as shown in FIG. 3 (IV: 3S,6S; III: 3R,6R; I: 3S,6R; II: 3R,6S). These peaks were not observed in the FMOC/HPLC analysis of the negative control (analogously set-up and incubated reaction mixture, but without alcohol dehydrogenase). Moreover, after a reaction time of 96 h, further product peaks were obtained at retention times of 37.479 min and 38.135 min. With reference to the standards, they can be assigned to the diamines 2,5-diamino-1,4:3,6-dianhydro-2,5-dideoxy-L-iditol (DAI) and 2,5-diamino-1,4:3,6-dianhydro-2,5-dideoxy-D-sorbitol (DAS).

Therefore coupling the three enzymatic reactions makes possible a net conversion rate of isosorbitol to the (3S,6S)-6-amino-3-alcohol as the main product with recycling factors of 30 for NADP⁺ and of 12 for L-alanine (FIG. 4).

1. A cell comprising
a polypeptide comprising the amino acid sequence of the Bacillus subtilis alanine dehydrogenase or a variant thereof, where Leu197 or an amino acid located at a homologous position in the amino acid sequence is exchanged for an amino acid with a positively charged side chain,
or a nucleic acid molecule comprising a nucleotide sequence encoding the polypeptide.

2. A process for the production of alanine or of a compound generated with consumption of alanine, comprising
c) reacting pyruvate with ammonium and NADPH to give alanine by contacting the pyruvate with the cell according to claim 1 or
with a polypeptide comprising the amino acid sequence of the Bacillus subtilis alanine dehydrogenase or a variant thereof, where Leu197 or an amino acid located at a homologous position in the amino acid sequence is exchanged for an amino acid with a positively charged side chain.

3. The process according to claim 2, further comprising
a) reacting a primary or secondary alcohol thereby producing an aldehyde or ketone by contacting the alcohol with an NADP⁺-dependent alcohol dehydrogenase in the presence of NADP⁺.

4. The process according to claim 3, further comprising
b) reacting of the aldehyde or ketone from a) to give an amine by contacting the aldehyde or ketone with a transaminase in the presence of alanine.

5. The process according to claim 4, wherein
a), b) and c) proceed in the same reaction mixture.

6. The process according to claim 3, wherein the cell further expresses an NADP⁺-dependent alcohol dehydrogenase.

7. The process according to claim 3, wherein the cell further expresses a transaminase.

8. The process according to claim 4, wherein, in the cell, all enzymes comprising the polypeptide, the NADP⁺-dependent alcohol dehydrogenase and the transaminase are expressed intracellularly in a localized manner.

9. The process according to claim 6, wherein the NADP⁺-dependent alcohol dehydrogenase is an NADP⁺-dependent alcohol dehydrogenase selected from the group consisting of the alcohol dehydrogenases from E. coli (YjgB, database code ZP_{—}07117674, a YahK, database code BAE76108.1), the alcohol dehydrogenase from Ralstonia sp. (SEQ ID No. 10), and variants thereof.

10. The process according to claim 7, wherein the transaminase is a transaminase selected from the group consisting of the transaminases from Chromobacterium violaceum (database code NP_{—}901695), Pseudomonas putida (database code YP_{—}001668026), Pseudomonas putida (database code YP_{—}001668026.1 or YP_{—}001671460), Rhodobacter sphaeroides (strain ATCC 17023; database code YP_{—}353455) and variants thereof.

11. The cell according to claim 1, wherein the amino acid with positively charged side chain is arginine.

12. The cell according to claim 1, wherein Asp196 or an amino acid located at a homologous position in the amino acid sequence is exchanged for an amino acid with a neutral or positively charged side chain.

13. The cell according to claim 1, wherein the amino acid with a neutral or positively charged side chain is an amino acid from the group consisting of alanine, glycine, serine and cysteine.

14. The cell according to claim 1, wherein the polypeptide is the polypeptide shown in SEQ ID No. 1.

15. The cell according to claim 1, wherein the nucleotide sequence is the nucleotide sequence shown in SEQ ID No. 2.

16. The cell according to claim 1, wherein the nucleic acid molecule is a vector comprising the nucleic acid molecule.
